(19) United States
(12) Patent Application Publication
(10) Pub. No.: US 2026/0345390 A1
(43) Pub. Date: Nov. 5, 2026

(54) AUTOMATED DOCKING OF AN OPHTHALMIC SYSTEM TO AN EYE

(71) Applicant: ALCON INC., Fribourg (CH)

(72) Inventors: Parisa RABBANI, Aliso Viejo, CA (US); Lingfeng YU, Rancho Santa Margarita, CA (US)

(21) Appl. No.: 19/658,949

(22) Filed: Apr. 27, 2026

Related U.S. Application Data
(60) Provisional application No. 63/800,116, filed on May 5, 2025.

(51) Int. Cl. A61F 9/008; G06T 7/00

(52) U.S. Cl. CPC A61F 9/008; G06T 7/0012; G06T 2207/30041

(57) ABSTRACT

In at least one embodiment, an ophthalmic system includes a laser system, an adjustment system, an imaging system, and a computer system. The laser system has an optical axis. A z-axis is defined relative to the optical axis, and an xy-plane, with a central xy-point, is defined relative to the z-axis. The imaging system generates an eye image set comprising images of an eye. The computer system determines an xy-offset of the eye according to the eye image set, where the xy-offset of the eye is relative to the central xy-point. The computer system also determines a z-distance to the eye according to the eye image set, where the z-distance corresponds to the z-axis. The computer system then instructs the adjustment system to compensate for the xy-offset of the eye and to facilitate movement of the optical head towards the eye according to the z-distance.

### FIELD

Embodiments of the present disclosure relate to automated docking of an ophthalmic system to an eye to perform an ophthalmic procedure on the eye.

### BACKGROUND

An ophthalmic system may have a laser system with an optical head that provides a laser beam to perform a procedure on a patient’s eye. The ophthalmic system may dock the optical head to the eye to hold the eye in place during the procedure. Correctly aligning the optical head with the eye during docking is important for the safety and effectiveness of the procedure.

### SUMMARY

In at least one embodiment, an ophthalmic system includes a laser system, an adjustment system, an imaging system, and a computer system. The laser system provides a laser beam. The laser system includes an optical head with an optical axis. The laser system is associated with a z-axis and an xy-plane, where the z-axis is defined relative to the optical axis, and the xy-plane is defined relative to the z-axis. The xy-plane has a central xy-point. The adjustment system facilitates movement of the optical head. The imaging system generates an eye image set comprising one or more images of an eye. The computer system determines an xy-offset of the eye according to the eye image set, where the xy-offset of the eye is relative to the central xy-point. The computer system also determines a z-distance to the eye according to the eye image set, where the z-distance to the eye corresponds to the z-axis. The computer system then instructs the adjustment system to compensate for the xy-offset of the eye and to facilitate the movement of the optical head towards the eye according to the z-distance to the eye.

One or more embodiments include none, one, two or more, most, or all of the following:
The computer system determines the xy-offset of the eye according to the eye image set by identifying an eye center according to the eye image set and determining a difference between the eye center and the central xy-point. The computer system may identify the eye center in any suitable manner. As an example, the computer system may identify the eye center by identifying a pupil according to the eye image set and identifying a pupil center of the pupil as the eye center. As another example, the computer system may identify the eye center by identifying a lens capsule according to the eye image set and identifying a lens capsule center of the lens capsule as the eye center. As yet another example, the computer system may identify the eye center by identifying corneal angles according to the eye image set, where a corneal angle is defined relative to a posterior surface of a cornea of the eye and an anterior surface of an iris of the eye, and identifying an angle center of the corneal angles as the eye center. As yet another example, the computer system may identify the eye center by determining an eye axis according to the eye image set and determining the eye center using the eye axis. As yet another example, the computer system may identify the eye center by receiving a selection of the eye center and identifying the selection as the eye center.
The computer system operates in a docking mode selected from a one-step docking process or a two-step docking process. The one-step docking process starts with a first patient interface coupled to the optical head, and the two-step docking process starts with a second patient interface coupled to the eye. The computer system may receive a selection the one-step docking process or the two-step docking process and operate according to the selection. The computer system may detect that a patient interface is coupled to the optical head and operate according to the one-step docking process. The computer system may detect that no patient interface is coupled to the optical head and operate according to the two-step docking process.
The computer system operates in a docking mode comprising a one-step docking process, the one-step docking process starting with a patient interface coupled to the optical head. The computer system may determine a contact of the patient interface with the eye according to the eye image set and instruct the adjustment system to reduce of stop the movement of the optical head according to the contact of the patient interface with the eye.
The computer system operates in a docking mode comprising a two-step docking process, the two-step docking process starting with a patient interface coupled to the eye. The computer system may determine a contact of the optical head with the patient interface according to the eye image set and instruct the adjustment system to reduce or stop the movement of the optical head according to the contact of the optical head with the patient interface.
The ophthalmic system includes a sensor that detects when the optical head is docked to the eye.
The ophthalmic system includes a sensor that detects a proximity of the patient interface to the eye.
The ophthalmic system includes a sensor that detects a proximity of the optical head to the patient interface.
The ophthalmic system includes a display device that displays a graphical user interface. The graphical user interface may display docking mode options, receive a selection of a docking mode selected from the docking mode options, and notify the computer system of the selection of the docking mode. The graphical user interface may display an automated docking selector, receive a selection of the automated docking selector, and notify the computer system of the selection of the automated docking selector.
The ophthalmic system includes a manual actuator that receives a selection of an automated docking option and notifies the computer system of the selection of the automated docking option.
The computer system instructs the adjustment system to compensate for the xy-offset of the eye according to a z-distance to the eye.
The computer system instructs the adjustment system to compensate for the xy-offset of the eye according to a predicted docking region.
The computer system instructs the adjustment system to compensate for the xy-offset of the eye according to a previous movement of the eye.

### BRIEF DESCRIPTION OF THE DRAWINGS

FIG. 1 illustrates an example of an ophthalmic system that may perform a procedure on an eye of a patient, according to at least one embodiment described in the present disclosure;

FIGS. 2A and 2B illustrate another example of an ophthalmic system that may perform a procedure on an eye, according to at least one embodiment described in the present disclosure;

FIG. 3 illustrates an example of a computing system, according to at least one embodiment described in the present disclosure;

FIGS. 4A and 4B illustrate an example of a one-step docking process, according to at least one embodiment described in the present disclosure;

FIGS. 5A and 5B illustrate an example of a two-step docking process, according to at least one embodiment described in the present disclosure;

FIGS. 6A and 6B illustrate examples of images of an eye that may be presented on a display device, according to at least one embodiment described in the present disclosure;

FIGS. 7A -7C illustrate an example of determining a z-distance to an eye using images of the eye, according to at least one embodiment described in the present disclosure;

FIG. 8 illustrates an example of a graphical user interface (GUI) presented on a display device, according to at least one embodiment described in the present disclosure; and

FIG. 9 illustrates an example of a method to perform a docking process, according to at least one embodiment described in the present disclosure.

### DESCRIPTION OF EXAMPLE EMBODIMENTS

Referring now to the description and drawings, one or more example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

The present disclosure relates to embodiments of an ophthalmic system that docks a laser optical head to an eye in an automated manner to perform a procedure on the eye. In one or more embodiments, the ophthalmic system determines that the eye is not centered and adjusts the laser optical head to center the eye. In one or more embodiments, the ophthalmic system determines a distance between the laser optical head and the eye and moves the laser optical head towards the eye according to the distance.

Certain embodiments of the present disclosure may provide improvements over known ophthalmic systems. For example, an ophthalmic system automatically adjusts a laser optical head to center the laser optical head with an eye. As another example, the ophthalmic system moves the laser optical head towards the eye according to a measured distance to the eye. As yet another example, the ophthalmic system may operate in a selected docking mode of multiple docking mode options. These and other features may improve the effectiveness, efficiency, and/or safety of the ophthalmic system.

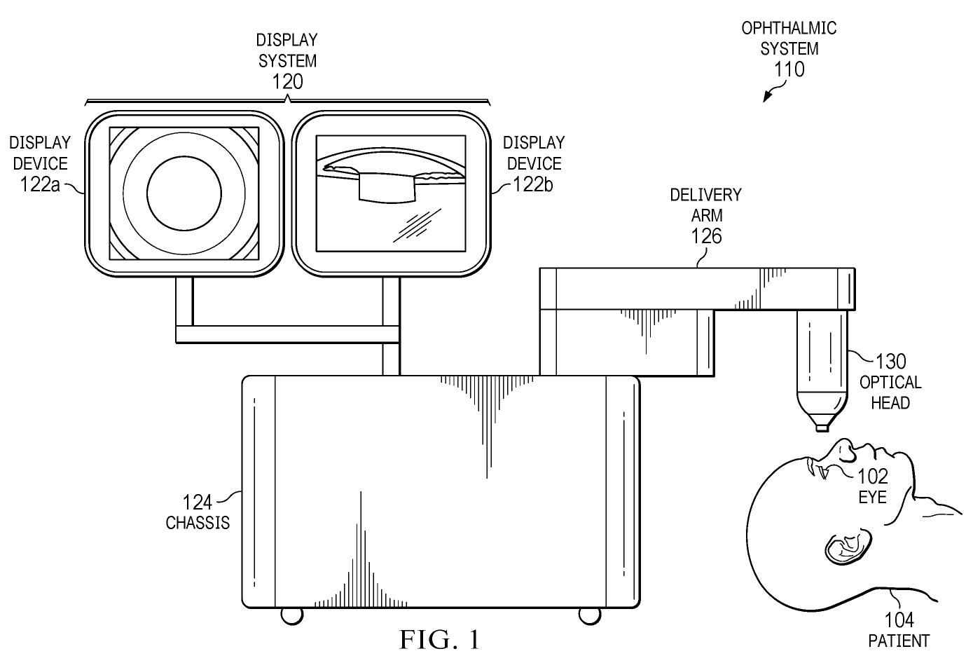

FIG. 1 illustrates an example of an ophthalmic system 110 that may perform a procedure on an eye 102 of a patient 104, according to at least one embodiment described in the present disclosure. In the example, the ophthalmic system 110 includes a display system 120 (with display devices 122 (122a and/or 122b)), a chassis 124, a delivery arm 126, and/or an optical head 130, which may be coupled (e.g., mechanically, electromagnetically, optically, communicatively, and/or otherwise coupled, such as with a vacuum coupling) as shown.

The ophthalmic system 110 may be any suitable medical device that performs a medical procedure, such as a surgical and/or diagnostic procedure. Examples of medical procedures include refractive, cataract, retinal, or other procedure performed on any suitable part of the eye 102. In one or more embodiments, the ophthalmic system 110 may be a surgical system that directs a laser beam to a part of the eye (e.g., cornea, lens, and/or other eye tissue) to process (e.g., ablate and/or photodisrupt) the tissue. In one or more embodiments, the ophthalmic system 110 may be a surgical system that performs a cataract surgery (e.g., a femtosecond laser assisted cataract (FLAC) surgery) to remove a crystalline lens from the eye.

In a FLAC surgery, a laser (e.g. a femtosecond laser) may be used to create photodisruptions in any suitable part of an eye. For example, a laser may create photodisruptions arranged in a fragmentation pattern to segment a crystalline lens of the eye to facilitate removal of the crystalline lens. As another example, a laser may create photodisruptions that form an incision in a lens capsule to perform a capsulotomy that provides access to the crystalline lens. In other examples, photodisruptions may be created to form one or more segments and/or one or more incisions (e.g., limbal relaxing incisions) in any suitable part of an eye.

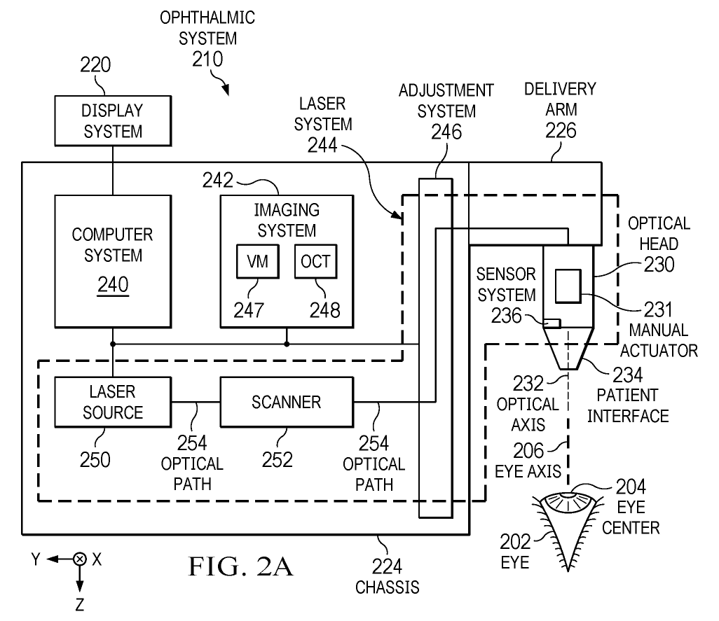

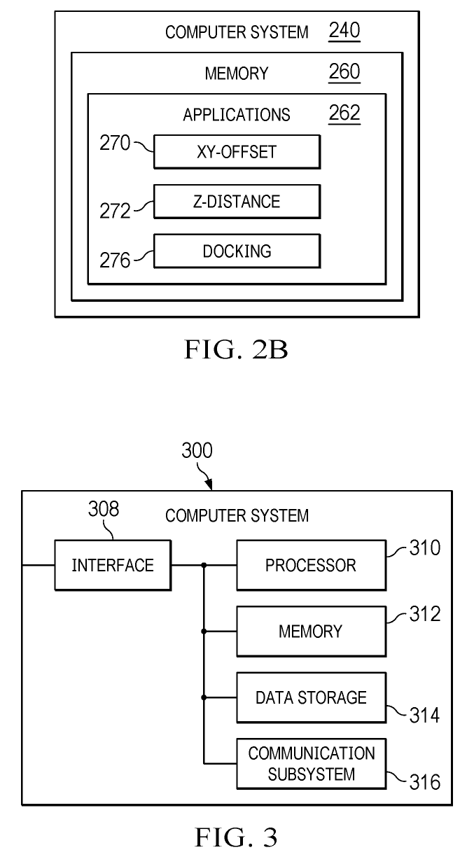

FIGS. 2A and 2B illustrate another example of an ophthalmic system 210 that may perform a procedure on an eye 202, according to at least one embodiment described in the present disclosure. In the example, the eye 204 has an eye center 204 and an eye axis 206. The ophthalmic system 210 includes a display system 220, a chassis 224, a delivery arm 226, an optical head 230 (with an optical axis 232), a computer system 240, an imaging system 242, a laser system 244, a manual actuator 231, a sensor system 236, and/or a patient interface 234, which may be coupled as shown. The imaging system 242 includes a video microscope (VM) system 247 and/or an optical coherence tomography (OCT) system 248. The laser system 244 includes a laser source 250, a scanner 252, an optical path 254, and/or an optical head 230, which may be coupled as shown. The computer system 240 has a memory 260 that stores applications 262, such as an xy-offset application 270, a z-distance application 272, and/or a docking application 276.

For ease of explanation, the embodiments may be described using the following example xyz-coordinate system, which may be regarded as the coordinate system of the laser system 244 and/ or the ophthalmic system 210, although any suitable coordinate system may be used. In the example, the z-axis is defined relative to the optical axis 232 of the laser system 244, e.g., the z-axis is aligned with the optical axis 232. A z-direction may be a direction along the z-axis. In the example, an xy-plane is orthogonal to the z-axis. For example, an xy-plane may be the target plane at which the target (e.g., the eye 202) is located when the target is aligned in the z-direction. The xy-plane may have a central xy-point at which the target should be centered.

As an overview of the example, the imaging system 242 generates an eye image set that includes one or more images of the eye 202. The computer system 240 determines an xy-offset of the eye according to the eye image set, where the xy-offset is relative to a central xy-point of an xy-plane. The computer system 240 also determines a z-distance to the eye according to the eye image set, where the z-distance is relative to the z-axis. The computer system 240 instructs the adjustment system 246 to compensate for the xy-offset of the eye. The computer system 240 also instructs the adjustment system 246 to move the optical head towards the eye 202 according to the z-distance. After the computer system 240 confirms that the eye 202 is properly docked, a procedure may begin, e.g., the laser system 244 may direct a laser beam towards the eye 202.

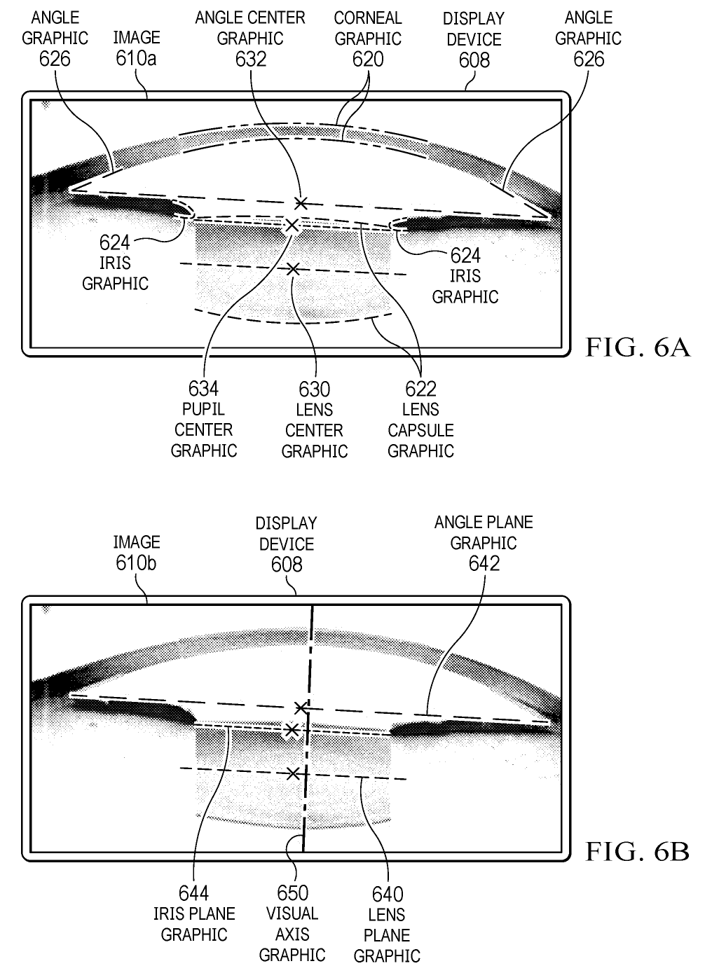

In the example, the eye 204 has an eye center 204 and an eye axis 206. The eye center 204 may be any suitable center of the eye 202. Examples of an eye center 204 include a capsule center, a pupil center, a limbus center, an angle center, a visual axis center, and/or other center of a structure of the eye, which are described herein, e.g., with reference to FIGS. 6A and 6B. An eye center may also be a function of one or more other eye centers. For example, an eye center may be a predefined distance from the pupil center or may be a predefined point between a pupil center and a limbus center.

The eye axis 206 may be any suitable axis of the eye 202. Examples of an eye axis 206 include a visual axis, an optical axis, and a pupil center axis, and/or other axis of a structure of the eye, which are described herein, e.g., with reference to FIGS. 6A and 6B. An eye axis may also be a function of one or more other eye axes. For example, an eye axis may be a predefined angle from the visual axis or may be a predefined line that is between a visual axis and an optical axis.

The eye 202 may have other suitable features, such as an eye plane. Examples of an eye plane include an iris plane, a lens plane, an angle plane, and/or other planar feature of a structure of the eye, which are described herein, e.g., with reference to FIGS. 6A and 6B. An eye plane may also be a function of one or more other eye planes. For example, an eye plane may include an average plane between the lens plane and the angle plane.

Turning to the components, the patient interface 234 couples the laser system 244 (e.g., the optical head 230 of the laser system 244) to the eye 202 to maintain the eye 202 in position during a procedure. The patient interface 234 may have a laser system end and a contact end. The laser system end may be coupled to the laser system 244 in any suitable manner, e.g., with a mechanical coupling, a vacuum coupling, and/or any other coupling approach. The contact end may be coupled to the eye 202 in any suitable manner, e.g., with a vacuum coupling. The patient interface 234 may include one or more parts, which may be coupled together in any suitable manner e.g., with a mechanical coupling and/or a vacuum coupling.

The patient interface 234 may begin the docking process coupled to the laser system 244 or to the eye 202, depending on the docking process. For example, in a one-step docking process, the patient interface 234 is coupled to the optical head 230 at the beginning of the process, and the ophthalmic system 210 lowers the optical head 230 until the patient interface 234 is in a proper docking position on the eye 202, e.g., a position where the patient interface 234 is ready to be coupled to the eye 202. As another example, in a two-step docking process, the patient interface 234 is coupled to the eye 202 (e.g., a user may hold the patient interface 234 in place on the eye 202) at the beginning of the process. The ophthalmic system 210 lowers the optical head 230 until the optical head 230 is in a proper docking position on the patient interface 234, e.g., a position where the optical head 230 is ready to be coupled to the patient interface 234.

The chassis 224 houses and/or supports at least a portion of the laser system 244, the imaging system 242, and/or the computer system 240. The chassis 224 is coupled to and/or supports the delivery arm 226. The delivery arm 226 facilitates movement of the optical head 230 to allow the optical head 230 to be positioned relative to the eye 202. In some embodiments, the delivery arm 226 may be cantilevered relative to the chassis 224. The adjustment system 246 moves the delivery arm 226. The adjustment system 246 may be any suitable gantry (e.g., a mechanical, electromagnetic, and/or hydraulic gantry) that can move the delivery arm 226 in response to instructions from the computer system 240 and/or a user. A user may use the manual actuator 231 (e.g., a joystick) to send instructions to, e.g., move the optical head 230 or initiate an automated docking process.

The laser system 244 directs a laser beam towards the eye 202. The laser system 244 may include a laser source 250 that generates the laser beam and a scanner 252 that guides the laser beam to points of the eye 202. The laser source may be a femtosecond laser or other ultrashort pulse laser. The laser beam may have any suitable pulse duration, such as in the order of nanoseconds, picoseconds, femtoseconds, or attoseconds. The laser beam may have any suitable wavelength, such as in the range of 150 nanometers (nm) to 20 micrometers (µm). Examples of ranges include the ultraviolet (e.g., in the range of 180 to 400 nm, such as 190 to 195 nm and/or 345 to 355 nm), visible, or infrared (including near-infrared) wavelength (e.g., in the range of 1000 to 1250 and/or 1250 to 1500 nm).

One or more optical devices direct the laser beam along the optical path 254 towards the eye 202. An optical device can act on (e.g., transmit, reflect, refract, diffract, collimate, condition, shape, focus, modulate, and/or otherwise act on) a laser beam. Examples of optical devices include a lens, prism, mirror, diffractive optical element (DOE), holographic optical element (HOE), and spatial light modulator (SLM).

The optical head 230 includes an objective lens that directs the laser beam towards the eye 202. The optical head 230 has an optical axis 232, which may be an optical axis of the objective lens. In one or more embodiments, the optical axis 232 may be used to align the eye 202 with the optical head 230.

The imaging system 242 generates an eye image set of images of the eye 202. The imaging system 242 may include one or more of any suitable systems that detect light reflected from the eye 202 to generate an image of the eye 202. In the example, the imaging system 242 includes a video microscope (VM) system 247 and/or an optical coherence tomography (OCT) system 248.

In one or more embodiments, the imaging system 242 may include an eye tracker that utilizes the video microscope system 247, the OCT system 248, and/or the computer system 240 to monitor movement of the eye 202 to, e.g., track an xy-offset and/or a z-distance to the eye 202 during a docking procedure. In the embodiments, the eye tracker may use, e.g., a stochastic approach, to analyze previous movements of the eye 202 to predict potential future movements of the eye 202. In one or more embodiments, the eye tracker may use previous movements of the eye 202 as the z-distance to the patient interface 234 decreases to predict a docking region within which the eye 202 may come into contact with the patient interface 234. For example, a stochastic approach may predict that there is a high probability (e.g., a probability that has a value within a range of, e.g., 50% to 75%, 75% to 90%, 90% to 95%, and/or 95% to 99%) that the eye 202 may come into contact with the patient interface 234 within the docking region.

The video microscope system 247 includes a microscope that receives light reflected from the eye 202 and uses the light to generate a microscope image of the eye 202. The microscope image may be an optical and/or digital image and may comprise one frame (e.g., a still image) or a series of frames (e.g., a video). In one or more embodiments, the microscope may receive the light through the optical head 230 to generate an on-axis image of the eye 202, which may be used for any suitable purpose. For example, a user may utilize a real-time on-axis image to position the optical head 230 over the eye 202. As another example, the computer system 240 may use an on-axis image to detect an eye center of the eye to determine an xy-offset of the eye 202.

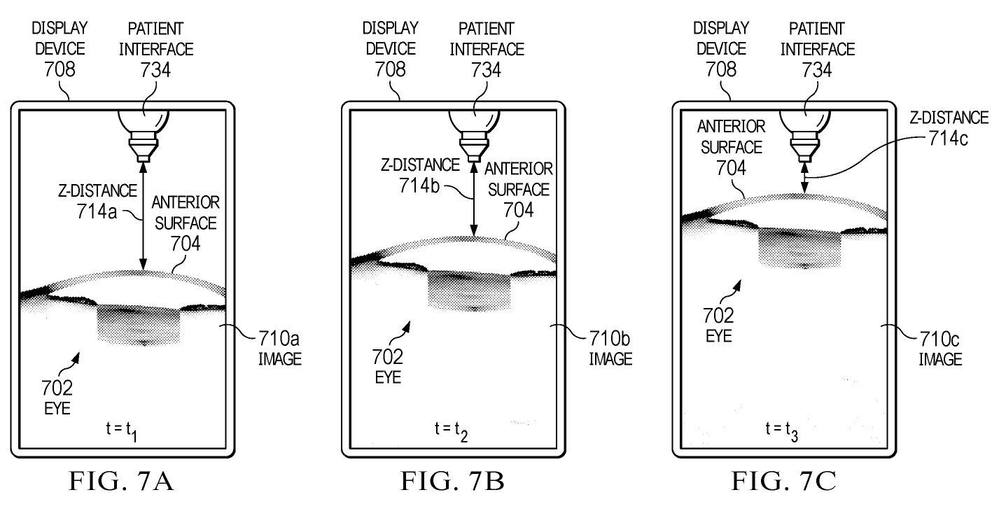

The OCT system 248 generates an OCT image of the eye 202. The OCT image may be a two-dimensional (2D) or a three-dimensional (3D) image. The OCT image may show any suitable portion of the eye 202. For example, the OCT image may show a cross-section of the eye 202, which may be used to determine an eye center and/or an eye axis, as described herein, e.g., with reference to FIGS. 6A and 6B. As another example, the OCT image may show a z-distance to the eye 202, as described herein, e.g., with reference to FIGS. 7A-7C. The OCT system 248 may share the same scanner 252 with the laser system 244 or may use a different scanner than the laser system 244 uses.

The display system 220 includes one or more display devices. Examples of display devices include computer monitors, display screens, or other devices that can display an image of the eye 202 and/or a graphical user interface (GUI). In one or more embodiments, the display system 220 may present a real-time microscope image of the eye 202, an OCT image of the eye 202, and/or a GUI.

The sensor system 236 may include one or more sensors that can be used by the computer system 240 to, e.g., identify a docking mode, determine a z-distance to the eye 202, and/or detect a proper docking position on the eye 202. Examples of sensors include pressure sensors, force sensors, contact sensors, temperature sensors, position sensors, proximity sensors, light sensors, color sensors, and/or other sensors.

In one or more embodiments, a sensor (e.g., of the sensor system 236) may be used to determine a z-distance to the eye 202. For example, a proximity sensor may be used to determine the z-distance. In one or more embodiments, a sensor (e.g., of the sensor system 236) may be used to detect a docking mode. For example, a contact sensor may detect whether the patient interface 234 is coupled to the optical head 230. In another example, a suction system may couple the patient interface 234 to the eye 202, and a sensor may detect whether the suction system is engaged. If one or more sensors detect that the patient interface 234 is coupled to the optical head 230 and/or that the suction system is not engaged, the computer system 240 may identify the docking mode as a one-step docking process. Otherwise, the computer system 240 may identify the docking mode as a two-step docking process. In other embodiments, the computer system 240 may determine whether the suction system is engaged or not engaged in any other suitable manner, e.g., the computer system 240 may control the suction system and may determine that it has or has not engaged the suction system.

In one or more embodiments, a sensor (e.g., of the sensor system 236) may be used to detect when the optical head 230 is in a proper docking position on the eye 202. In the embodiments, a pressure sensor, a force sensor, a displacement sensor, and/or other suitable sensor may indicate a pressure and/or a force with which the patient interface 234 is pressing against the eye 202. For example, in a one-step docking process, a pressure sensor and/or a force sensor may detect when the patient interface 234 (which is coupled to the optical head 230) is in a proper docking position on the eye 202. As another example, in a two-step docking process, a pressure sensor and/or a force sensor may detect when the optical head 230 is in a proper docking position on the patient interface 234 (which is coupled to the eye 202).

A pressure and/or a force with which the patient interface 234 is pressing against the eye 202 may be measured in any suitable manner. In one or more embodiments, measurements of the pressure and/or the force may be made at one or more stages of a docking procedure. The one or more stages may include, e.g., a float stage during which the patient interface 234 is floating atop of the eye 202, an optimal stage during which suction may be activated, and/or an end-of-travel stage during which the patient interface 234 should stop pressing against the eye 202. In one or more embodiments, the measurements may be checked at the one or more stages to see if they are acceptable. In an example, acceptable measurements may be (measured in units of g-force): 25 g to 75 g at a float stage, 130 g to 170 g at an optimal stage, and 250 g to 350 g at an end-of-travel stage.

In one or more embodiments, the sensor system 236 and/or the imaging system 242 may be configured to monitor the z-direction position of the patient interface 234 relative to the eye 202. In some embodiments, output from the sensor system 236 and/or the imaging system 242 may be used to indicate when a suction system should employ suction to fixedly couple the patient interface 235 to the eye 202. For example, when a sensor detects a threshold amount of force or pressure and/or the imaging system 242 indicates that the eye 202 is aligned with the optical head 230, the suction system may be engaged.

In some embodiments, a primary system may determine a z-direction position, and a secondary system may be used to check (e.g., validate and/or confirm) the z-direction position determined by the primary system. For example, the sensor system 236 may be used to check a z-direction position determined by the imaging system 242 (e.g., using OCT 248), and/or the imaging system 242 may be used to check a z-direction position determined by the sensor system 236. In some embodiments, one system may operate as the primary system that determines the z-direction position during one portion of a docking procedure, and another system may operate as the primary system that determines the z-direction position during another portion of the docking procedure. For example, the imaging system 242 may act as the primary system for determining the z-direction position until contact is made between the eye 202 and the patient interface 234, after which the sensor system 236 may act as the primary system for determining the z-direction position.

The computer system 240 performs operations for the ophthalmic system 210. The computer system 240 may send instructions to components of the ophthalmic system 210, e.g., the display system 220, the computer system 240, the imaging system 242, and/or the laser system 244. The computer system 240 may utilize applications 262 to perform the operations. In the example, the applications 262 include an xy-offset application 270, a z-distance application 272, and/or a docking application 276. The applications 262 may also include image processing software that can be used to analyze an image of an eye to, e.g., identify structures of the eye in the image.

The computer system 240 may use the xy-offset application 270 to determine the xy-offset in any suitable manner. For example, the computer system 240 may identify an eye center (which, in some but not all cases, may be indicated by an eye axis) in an image of the eye and determine the xy-offset according to the eye center and a central xy-point of an xy-plane, e.g., the target plane, as described herein, e.g., with reference to FIGS. 6A and 6B.

The computer system 240 may use the z-distance application 272 to determine the z-distance in any suitable manner. For example, the computer system 240 may identify an anterior surface in an image of the eye and determine the z-distance according to the anterior surface, as described herein, e.g., with reference to FIGS. 7A and 7B. As another example, the computer system 240 may use a sensor of sensor system 236 to determine the z-distance, as described above.

The computer system 240 uses the docking application 276 to perform a docking process. In an example, the computer system 240 instructs the adjustment system 246 to compensate for an xy-offset of the eye and/or to move the optical head 230 towards the eye 202 according to a z-distance to the eye 202 until the optical head 230 is in a proper docking position on the eye 202. In one or more embodiments, the instructions may instruct the adjustment system 246 to move in a specified direction (e.g., x-direction, y-direction, and/or z-direction) for a specified distance and/or at a specified rate.

An xy-offset of the eye 202 may be compensated for in any suitable manner. In one or more embodiments, the xy-offset may be compensated for relative to the z-distance to the eye 202. For example, for longer z-distances, the adjustment system 246 may move longer distances and/or at faster rates to compensate for the xy-offset. As the z-distance becomes shorter, the adjustment system 246 may move shorter distances and/or at slower rates to compensate for the xy-offset.

In one or more embodiments, the xy-offset may be compensated for according to previous movements of the eye 202. In some embodiments, the computer system 240 may regard the movements of the eye 202 as stable and thus not attempt to compensate for every single movement of the eye 202. The movements may be considered stable if, e.g., the movements are within a small region (e.g., within 3 millimeters (mm) away from the center) for a high percentage of movements (e.g., a percentage with a value within a range of 60% to 80% and/or 80% to 100%). In an example, if the eye 202 has moved only 0.5 mm away from the center for 75% of the eye movements during docking, the adjustment system 246 may not try to correct for a single instance of the eye moving 1 mm away from the center.

In one or more embodiments, the xy-offset may be compensated for according to a predicted docking region within which the eye 202 may come into contact with the patient interface 234. In an example, as the z-distance decreases, the size of the predicted docking region may become smaller. Accordingly, the corrections for the xy-offset may be coarser for larger z-distances (when the predicted docking region is larger) and may be more precise as the z-distance decreases (when the predicted docking region is smaller).

In one or more embodiments, the computer system 240 instructs the adjustment system 246 to move the optical head 230 towards the eye 202 according to a z-distance to the eye 202 until the optical head 230 is in a proper docking position on the eye 202. In some embodiments, the rate at which the optical head is moved towards the eye 202 may change according to the z-distance. For example, for larger z-distances, the rate of movement in the z-direction may be larger, and as the z-distance decreases (e.g., the optical head moves closer to the eye), the rate of movement may decrease. By decreasing the rate of movement in the z-direction as the patient interface comes closer to the eye, the system may be given sufficient time to correct for xy-offset discrepancies before docking/physical contact between the patient interface and the eye.

The proper docking position may be, e.g., when a patient interface 234 coupled to the optical head 230 is in contact with the eye 202 (in a one-step docking process) or when the optical head 230 is in contact with a patient interface 234 coupled to the eye 202 (in a two-step docking process). Once the optical head 230 is in a proper docking position, then the computer system 240 instructs a suction system to apply suction to couple the optical head 230 to the eye 202 via the patient interface 234. After the computer system 240 confirms that the eye 202 is properly docked, the procedure may start, e.g., the laser system 244 may direct a laser beam towards the eye 202.

The computer system 240 may use the docking application 276 to determine the docking mode, e.g., a one-step docking process or a two-step docking process. For example, the computer system 240 may present a graphical user interface that a user may use to input a selected docking mode. In one or more cases, a docking mode may be selected for a given procedure, a given surgeon, a given medical office, or other suitable given condition. In one or more cases, a docking mode may be selected as a default docking mode for the given condition. The computer system 240 may receive the selected docking mode and operate according to the selected docking mode. As another example, the computer system 240 may use the sensor system 236 to detect whether the patient interface 234 is coupled to the optical head 230. If the sensor system 236 detects that the patient interface 234 is coupled to the optical head 230 and not in contact with the eye 202, the computer system 240 may determine the docking mode is the one-step docking process. If the sensor system 236 does not detect that the patient interface 234 is coupled to the optical head 230, the computer system 240 may determine that the docking mode is the two-step docking process.

The computer system 240 may use the docking application 276 to perform the one-step docking process and/or the two-step docking process. For example, in the one-step docking process, the computer system 240 may instruct the adjustment system 246 to move the optical head 230 (with a patient interface 234) towards the eye 202 until the patient interface 234 is in a proper docking position on the eye 202. The computer system 240 may analyze images from the imaging system 242 and/or information from the sensor system 236 to determine the z-distance to the eye 202 and the docking position of the patient interface 234 on the eye 202. The computer system 240 may instruct the adjustment system 246 to reduce movement of the optical head 230 towards the eye 202 as the patient interface 234 comes closer to the eye 202 and stop the movement when the patient interface 234 is in a proper docking position on the eye 202.

As another example, in the two-step docking process, the computer system 240 may instruct the adjustment system 246 to move the optical head 230 towards a patient interface 234 on the eye 202 until the optical head 230 is in a proper docking position on the patient interface 234. The computer system 240 may analyze images from the imaging system 242 and/or information from the sensor system 236 to determine the z-distance to the eye 202 and the docking position of the optical head 230 on the patient interface 234. The computer system 240 may instruct the adjustment system 246 to reduce movement of the optical head 230 towards the eye 202 as the optical head 230 comes closer to the patient interface 234 and stop the movement when the optical head 230 is in a proper docking position on the patient interface 234 on the eye 202.

FIG. 3 illustrates an example of a computing system 300, according to at least one embodiment described in the present disclosure. The computing system 300 may include an interface 308, a processor 310, a memory 312, a data storage 314, and/or a communication subsystem 316, any or all of which may be communicatively coupled. Any or all of the computing system 300 may be implemented as computer hardware and/or software. Any or all of the computer system described herein may be implemented as a computing system consistent with the computing system 300.

In the example, the interface 308 may receive input to the computing system 300 and/or send output from the computing system 300, and may be used to exchange information between, e.g., software, hardware, one or more peripheral devices, one or more users, and/or any suitable combinations of any of the preceding. A user interface is a type of interface that a user can utilize to communicate with (e.g., send input to and/or receive output from) the computing system 300. Examples of user interfaces include displays, Graphical User Interfaces (GUIs), touchscreens, foot pedals, keyboards, computer mouses (or mice), gesture sensors, microphones, and speakers.

Generally, the processor 310 may include any suitable special-purpose or general-purpose computer, computing entity, or processing device including various computer hardware or software modules and may be configured to execute instructions stored on any applicable computer-readable storage media. For example, the processor 310 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. Although illustrated as a single processor in FIG. 3, the processor 310 may include any number of processors distributed across any number of network or physical locations that are configured to perform individually or collectively any number of operations described in the present disclosure.

The processor 310 may perform any suitable operations. In some embodiments, the processor 310 may interpret and/or execute program instructions and/or process data stored in the memory 312, the data storage 314, or the memory 312 and the data storage 314. In some embodiments, the processor 310 may fetch program instructions from the data storage 314 and load the program instructions into the memory 312. After the program instructions are loaded into the memory 312, the processor 310 may execute the program instructions, such as instructions to perform any of the methods disclosed herein, respectively.

The memory 312 and the data storage 314 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 310.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 310 to perform a certain operation or group of operations.

The communication subsystem 316 may include any component, device, system, or combination thereof that is configured to transmit, receive, and/or otherwise exchange information over a network in order to communicate with any suitable entity, such as with other devices at other locations or at the same location or even within the same system. The communication subsystem 316 may provide for communication among the devices described in the present disclosure, communication networks, computing devices, and other systems. For example, the communication subsystem 316 may allow the computing system 300 to communicate with other systems, such as other computing devices and/or networks. In some embodiments, the communication subsystem 316 may include a modem, a network card (wireless or wired), an optical communication device, an infrared communication device, a wireless communication device (such as an antenna), and/or chipset. Examples of communication subsystem 316 include a Bluetooth device, an 802.6 device (e.g., that can communicate with a Metropolitan Area Network (MAN)), a WiFi device, a WiMax device, cellular communication facilities, and/or the like.

One skilled in the art will recognize that modifications, additions, or omissions may be made to the computing system 300 without departing from the scope of the present disclosure. For example, the computing system 300 may include more or fewer components than those explicitly illustrated and described.

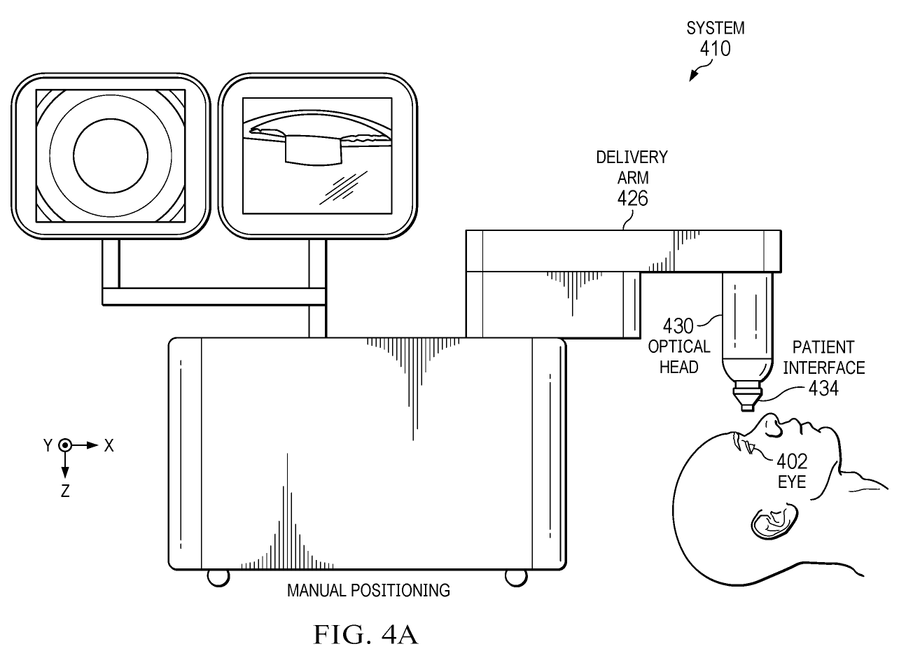

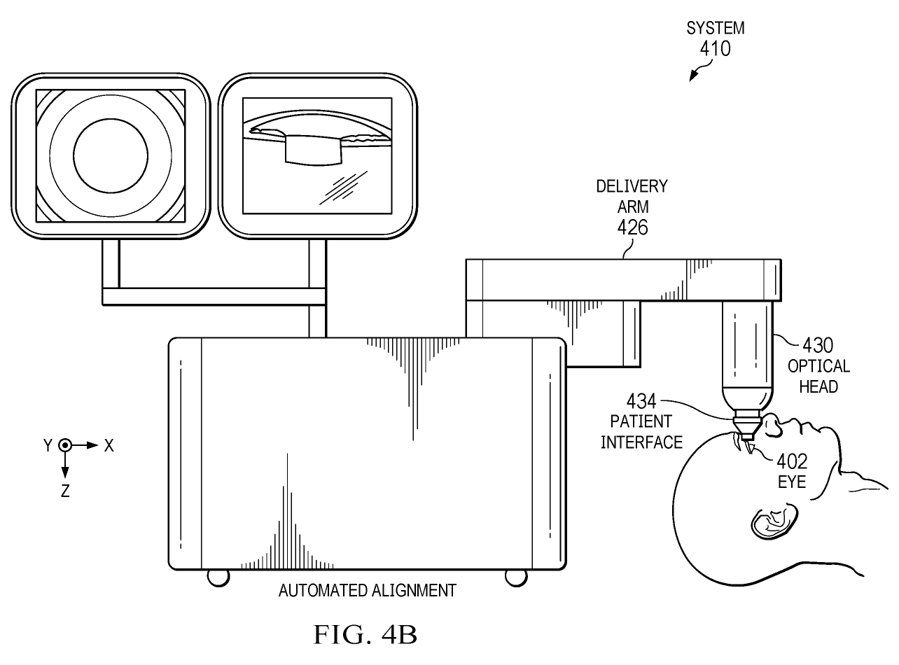

FIGS. 4A and 4B illustrate an example of a one-step docking process, according to at least one embodiment described in the present disclosure. In the example, an ophthalmic system 410 includes a delivery arm 426, an optical head 430, and/or a patient interface 434. In the one-step docking process, the patient interface 434 is coupled to the optical head 430, and the ophthalmic system 410 lowers the optical head 430 until the patient interface 434 is in a proper docking position on the eye 402. In the example, a manual positioning process is used to, e.g., make a coarse adjustment to approximately position the optical head 430 proximate to the eye 402. Then, an automated alignment process is used to, e.g., make a fine adjustment to dock the optical head 430 (with the patient interface 434) onto the eye 402.

FIG. 4A describes an example of the manual positioning process of the optical head 430. In the example, a user positions the optical head 430 above the eye 402. The user may utilize a manual actuator (e.g., a joystick) to manually position the optical head 430. An imaging system of the ophthalmic system 410, such as a video microscope system, may assist the user with aligning the optical head 430 relative to the eye 402.

FIG. 4B describes an example of the automated alignment process of the optical head 430 that may be performed by a computer system of the ophthalmic system 410. The user may initiate the automated alignment process by engaging and/or disengaging a manual actuator, e.g., pressing and/or releasing a button of a joystick and/or pressing and/or releasing a foot pedal switch. In response, the computer system may confirm via a video microscope system that the eye 502 is within a range of the optical head 530, e.g., centered and within, e.g., a 10 centimeters range of the optical head in a z-direction. The computer system may then instruct an adjustment system of the ophthalmic system 410 to lower the optical head 430 until the patient interface 434 is in a proper docking position on the eye 402, as described herein, e.g., with reference to FIG. 2A.

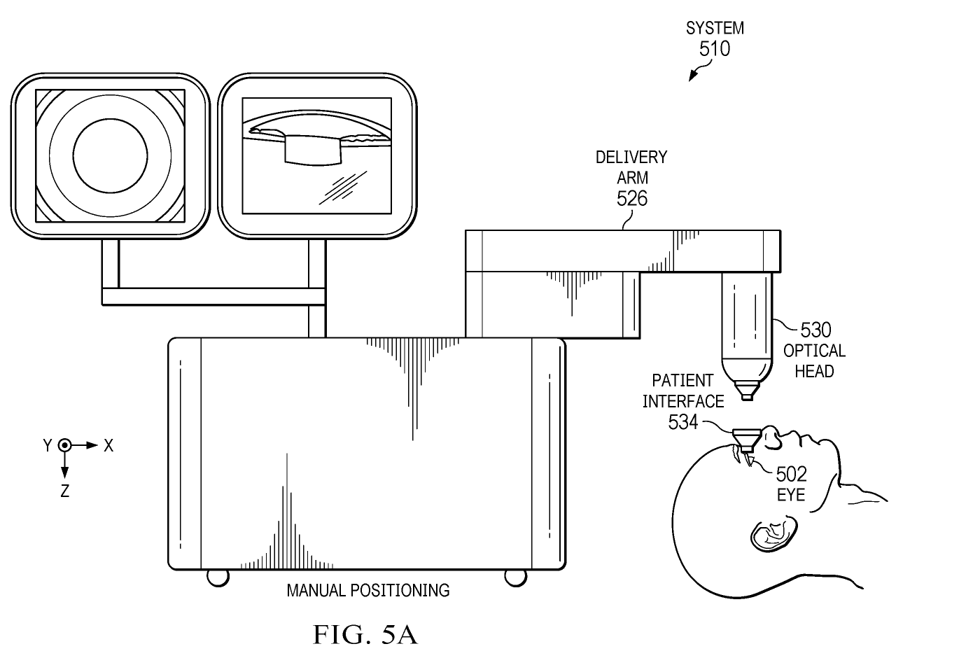

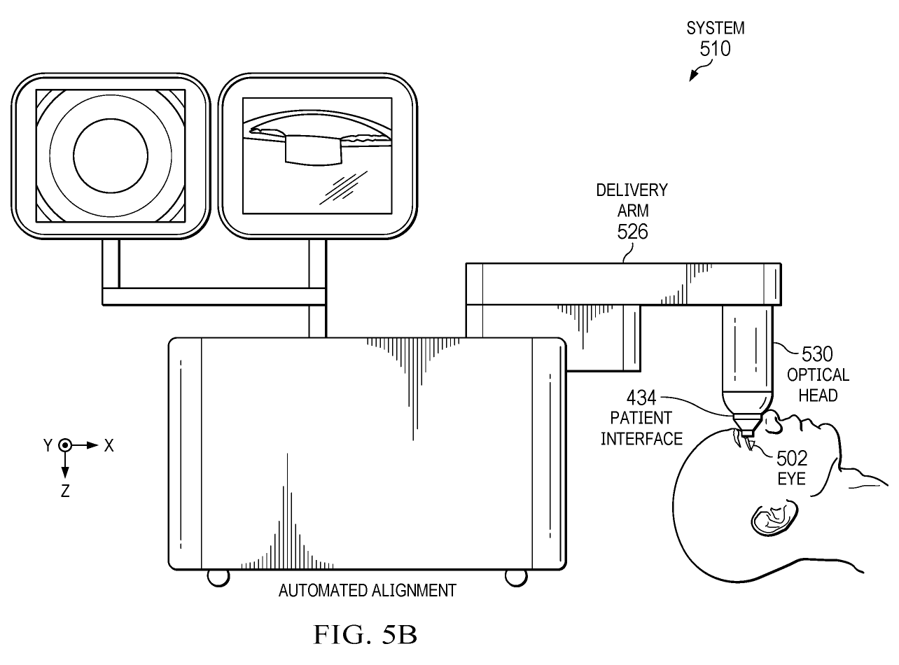

FIGS. 5A and 5B illustrate an example of a two-step docking process, according to at least one embodiment described in the present disclosure. In the example, an ophthalmic system 510 has a delivery arm 526, an optical head 530, and/or a patient interface 534. In the two-step docking process, the patient interface 534 is coupled to the eye 502, e.g., a user holds the patient interface 534 in position on the eye 502 and then engages a suction system to couple the patient interface 534 to the eye 502. The ophthalmic system 510 lowers the optical head 530 until the optical head 530 is in a proper docking position on the patient interface 534 on the eye 502. In the example, a manual positioning process is used to, e.g., make a coarse adjustment to approximately position the optical head 530 proximate to the eye 502. Then, an automated alignment process is used to, e.g., make a fine adjustment to dock the optical head 530 onto the patient interface 534 on the eye 502.

FIG. 5A describes an example of the manual positioning process of the optical head 530. In the example, a user positions the optical head 530 above the eye 502. The user may utilize a manual actuator (e.g., a joystick) to manually position the optical head 530. An imaging system of the ophthalmic system 510, such as a video microscope system, may assist the user with aligning the optical head 530 relative to the eye 502.

FIG. 5B describes an example of the automated alignment process of the optical head 530 that may be performed by a computer system of the ophthalmic system 510. The user may initiate the automated alignment process by engaging and/or disengaging a manual actuator, e.g., pressing and/or releasing a button of a joystick and/or pressing and/or releasing a foot pedal switch. In response, the computer system may confirm via a video microscope system that the eye 502 is within a range of the optical head 530, e.g., centered and within, e.g., a 10 centimeters range of the optical head in a z-direction. The computer system may then instruct an adjustment system of the ophthalmic system 510 to lower the optical head 530 until the optical head 530 is in a proper docking position on the patient interface 534 on the eye 502, as described herein, e.g., with reference to FIG. 2A.

FIGS. 6A and 6B illustrate examples of images 610 (610a and 610b, respectively) of an eye that may be presented on a display device 608, according to at least one embodiment described in the present disclosure. The display device 608 may be a part of any suitable ophthalmic system, as described herein. An image 610 (610a and/or 610b) may include an OCT image of the eye and one or more eye structure graphics that indicate structures of the eye (e.g., a cornea, a lens, and/or an iris) in the OCT image. An image 610 (610a and/or 610b) may also include one or more geometric feature graphics that indicate one or more geometric features that a computer system can determine from structures of the eye. In one or more embodiments, the graphics may include one or more overlays or other markings of any suitable size, shape, and/or color. For example, the lens center graphic 630, the angle center graphic 632, and/or the pupil center graphic 634 may include an “X” overlayed onto the image 610. In the embodiments, the graphics may include a line may have any suitable line-style, such as any suitable thickness, color, and/or pattern (e.g., solid, dashed, and/or dotted). For example, the lens capsule graphic 622 may include one or more lines overlayed onto the image 610, where the line is shown as dashed.

An image 610 (610a and/or 610b) may include any suitable eye structure graphic that indicates any suitable part of any suitable structure of the eye. In the example, a cornea graphic 620 indicates an anterior surface and/or a posterior surface of the cornea. A lens capsule graphic 622 indicates an anterior surface and/or a posterior surface of the lens capsule. An iris graphic 624 indicates an interior circumference of the iris. An angle graphic 626 indicates a corneal angle between a posterior surface of the cornea and an anterior surface of the iris.

An image 610 (610a and/or 610b) may include any suitable geometric feature graphic, e.g., an eye center graphic, an eye plane graphic, and/or an eye axis graphic. In the example, the eye center graphics include a lens center graphic 630, an angle center graphic 632, and/or a pupil center graphic 634. In the example, the eye plane graphics include a lens plane graphic 640, an angle plane graphic 642, and/or an iris plane graphic 644. In the example, the eye axis graphic is a visual axis graphic 650.

A computer system may identify an eye center of an eye in any suitable manner. Examples of an eye center 204 include a capsule center, a pupil center, a limbus center, an angle center, a visual axis center, and/or other central structure of the eye. In one or more embodiments, the computer system detects a structure of the eye and then identifies a midpoint of the structure as an eye center. For example, the computer system detects a pupil (indicated by the iris graphic 624) and then identifies a midpoint of the pupil as a pupil center (indicated by the pupil center graphic 634). As another example, the computer system detects a lens capsule (indicated by the lens capsule graphic 622) and then identifies a midpoint of the lens capsule as a lens capsule center (indicated by the lens center graphic 630). As another example, the computer system detects corneal angles (indicated by the angle graphic 626) and then identifies a midpoint between the vertices of the corneal angles as an angle center (indicated by an angle center graphic 632). In one or more embodiments, the computer system detects an eye axis (as described herein, e.g., with reference to FIGS. 6A and 6B) and an eye plane (as described herein, e.g., with reference to FIGS. 6A and 6B) and then identifies a point where the eye axis intersects the eye plane as an eye center.

A computer system may identify an eye plane of an eye in any suitable manner. Examples of an eye plane include an iris plane, a lens plane, an angle plane, and/or other planar feature of a structure of the eye. In one or more embodiments, the computer system detects a structure of the eye and then identifies a plane of the structure as an eye plane. In the embodiments, the eye plane may be the plane defined by the structure of the eye. For example, an iris plane (indicated by the iris plane graphic 644) may be a plane defined by the iris (indicated by the iris graphic 624). As another example, a lens plane (indicated by the lens plane graphic 640) may be a plane that substantially bisects the lens (indicated by the lens capsule graphic 622) such that the plane is substantially parallel to the pupil plane. As another example, an angle plane (indicated by the angle plane graphic 642) may be a plane defined by the vertices of the corneal angles (indicated by the angle graphic 626).

A computer system may identify an eye axis of an eye in any suitable manner. Examples of an eye axis 206 include a visual axis, an optical axis, and a pupil center axis, and/or other axis of a structure of the eye. In one or more embodiments, the computer system detects features of one or more structure of the eye and then identifies a line through the features as an eye axis. In the embodiments, the eye axis may be the line through the features of the eye. For example, a visual axis may be a line through the corneal vertex (the highest point of the cornea) and the nodal point (the center of the corneal curvature), which may be identified using the cornea graphic 620. As another example, a visual axis may be a line through the fixation point and the fovea of the eye, which may also be identified in an image of the back of the eye (not shown). As another example, an optical axis may be a line orthogonal to a cornea surface (indicated by the cornea graphic 620) that intersects the pupil center (indicated by the pupil center graphic 634). As yet another example, an optical axis may be a line normal to at least two surfaces of the eye, e.g., the anterior and/or posterior surfaces of the cornea and/or the lens, which may be identified using the cornea graphic 620 and/or the lens capsule graphic 622. As yet another example, a pupillary axis may be a line that connects pupil center (indicated by the pupil center graphic 634) and the nodal point (the center of the corneal curvature), which may be identified using the cornea graphic 620.

FIGS. 7A-7C illustrate an example of determining a z-distance 714 (714a to 714c, respectively) to an eye using images 710 (710a to 710c, respectively) of the eye, according to at least one embodiment described in the present disclosure. The images 710 may be displayed by a display device 708 of any suitable ophthalmic system, as described herein. In the example, the images 710 (710a to 710c) each include an OCT image of an eye 702 (with an anterior surface 704) and a graphic representing a patient interface 734. The images 710 (710a to 710c) show the patient interface 734 moving relative to the eye 702 at time t (t = t₁ to t₃, respectively), and the z-distance 714 (714a to 714c, respectively) between the patient interface 734 and the anterior surface 704 at time t (t = t₁ to t₃, respectively).

In the example, the image 710a shows the z-distance 714a at time t₁, and the image 710b shows the z-distance 714b at time t₂. The z-distance 714b is less than the z-distance 714a, indicating that the patient interface 734 is moving closer to the eye 702. The image 710cshows the z-distance 714c at time t₃. The z-distance 714cis less than the z-distance 714b, indicating that the patient interface 734 is moving even closer to the eye 702. At this point, a computer system may soon instruct an adjustment system to reduce or stop the movement of the patient interface 734 towards to the eye 702.

In some embodiments, the computing system may be calibrated such that a number of pixels or other units of the image 710 may correspond to a given physical distance such that by measuring the z-distance 714 in the image 710, a corresponding physical distance may be determined. In some embodiments, such a calibration may be validated in a startup routine or other system check routine. Additionally or alternatively, the distance calibration may be performed manually. In some embodiments, the z-distance 714 may be measured based on another sensor type, such as an accelerometer or position sensor associated with the patient interface 734 or optical head (not shown) which may indicate a physical location of the patient interface 734 relative to an initial starting point (e.g., the patient interface 734 is known to have moved 2 cm in the z-direction from an initial starting point). In these and other embodiments, a distance from the initial starting point to the anterior surface 704 may be known, estimated, input by the surgeon, or otherwise determined. For example, a measurement may be taken of the height of the patient’s head, or the distance from the anterior surface 704 to the back of the head, such that a distance from the initial starting point to the headrest is known, and is offset by the measured height from the headrest/back of the head to the anterior surface 704 of the eye.

While illustrated as showing an OCT view of the eye and the docking process, it will be appreciated that other or additional views may be shown. For example, the display device 708 may include a microscope image that shows a surface view of the eye 702 or a view along the optical axis of the optical head. In these and other embodiments, a picture-in-picture view, a side-by-side view, or any other arrangement of two views may be included of both the OCT and the microscope view on the display device 708. In some embodiments, which view is being shown on the display device 708 out of multiple potential views may shift automatically during the course of docking. For example, a microscope view with a picture-in-picture view of the OCT may be shown during docking until the patient interface 734 is contacting the anterior surface 704 of the eye, at which point the OCT view may become a main view and the microscope view may be shifted to a secondary view.

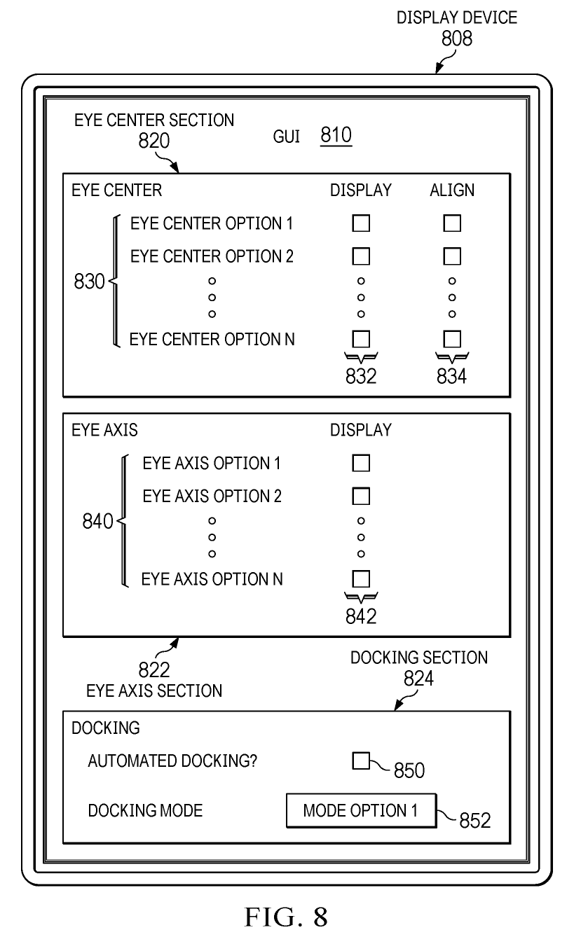

FIG. 8 illustrates an example of a graphical user interface (GUI) 810 presented on a display device 808, according to at least one embodiment described in the present disclosure. The display device 808 may be a part of any suitable ophthalmic system, as described herein. A user may use the GUI 810 to make one or more selections. In response, the GUI 810 receives the one or more selections and notifies a computer system of the ophthalmic system of the one or more selections.

In the example, the GUI 810 includes an eye center section 820, an eye axis section 822, and a docking section 824. The eye center section 820 includes eye center options 830, eye center display selectors 832, and eye center alignment selectors 834. The eye axis section 822 includes eye axis options 840 and eye axis display selectors 842. The docking section 824 includes an automated docking selector 850 and a docking mode selector 852.

The eye center section 820 includes the eye center options 830, display selectors 832, and/or align selectors 834. The eye center options 830 include Eye Center Option 1, Eye Center Option 2, … Eye Center Option N, where an Eye Center Option i, i = (1, 2, … N), represents an eye center as described herein, e.g., with reference to FIGS. 6A and 6B. The eye center options 830 may include a custom center option where a user may enter a customized eye center. For example, an eye center may include a function of one or more other eye centers, as described herein. Each eye center option 830 has a corresponding display selector 832 and/or alignment selector 834. A user may select a display selector 832 to display an eye center graphic that indicates the corresponding eye center. A user may select an align selector 834 to use the corresponding eye center to align the eye during a docking process.

The eye axis section 822 includes the eye axis options 840 and display selectors 842. The eye axis options 840 include Eye Axis Option 1, Eye Axis Option 2, … Eye Axis Option M, where an Eye Axis Option i, i = (1, 2, … M), represents an eye axis as described herein, e.g., with reference to FIGS. 6A and 6B. The eye axis options 840 may include a custom axis option where a user may enter a customized eye axis. For example, an eye axis may include a function of one or more other eye axes. Each eye axis option 840 has a corresponding display selector 842. A user may select a display selector 842 to display an eye axis graphic that indicates the corresponding eye axis.

The docking section 824 includes an automated docking selector 850 and/or a docking mode selector 852. A user may select an automated docking selector 850 to select an automated alignment process. A user may use the docking mode selector 852 to select the one-step docking process or the two-step docking process. In some embodiments, the docking mode selector 852 may include a default docking mode option.

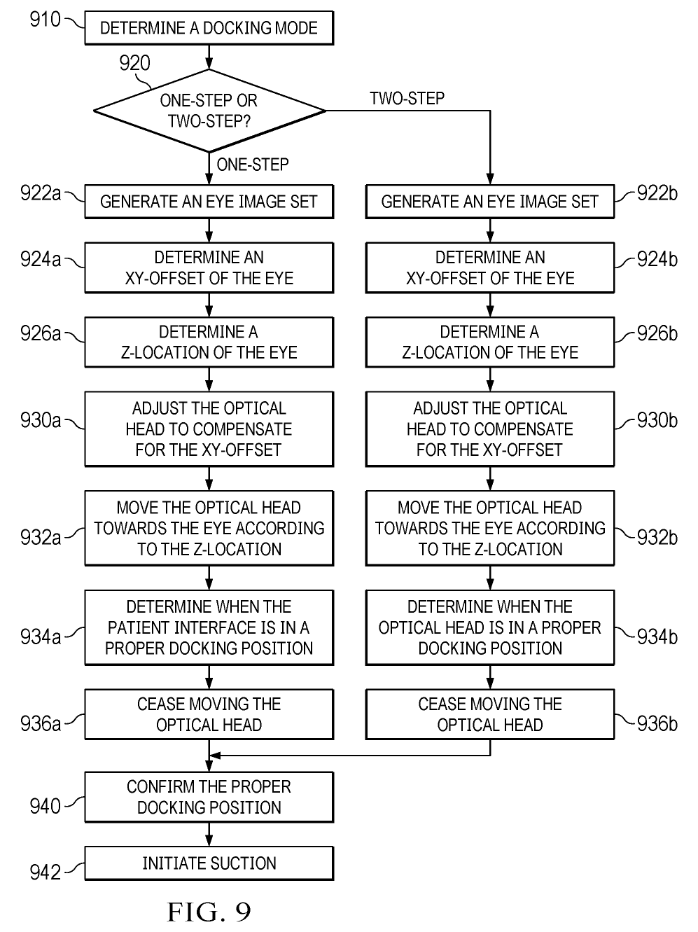

FIG. 9 illustrates an example of a method to perform a docking process, according to at least one embodiment described in the present disclosure. The method may be performed by any suitable system, such as an ophthalmic system described herein, to dock the system to an eye. In the example, the method is performed by an ophthalmic system that includes an imaging system, a computer system, and a laser system. The laser system may have an optical head with an optical axis that is used to define a z-axis and a central xy-point of the laser system.

At block 910, the computer system determines a docking mode. In the example, the docking mode may be a one-step docking process or a two-step docking process. The one-step docking process starts with a patient interface coupled to the optical head, and the two-step docking process starts with the patient interface coupled to the eye. In these and other embodiments, the determination may include establishing that the system is capable of both one-step and two-step docking and identifying which of the two is being used in the current procedure.

At block 920, if the docking mode is a one-step docking process, the method proceeds to block 922a.

At block 922a, the imaging system generates an eye image set that includes images of the eye. The images may include a microscope image of the eye and/or an OCT image of the eye.

At block 924a, the computer system determines an xy-offset of the eye according to the eye image set. For example, the computer system may identify an eye center (which, in some but not all cases, may be indicated by an eye axis) in the microscope image of the eye. The computer system may then determine the xy-offset from a difference between the eye center and the central xy-point of the laser system.

At block 926a, the computer system determines a z-distance to the eye according to the eye image set. For example, the computer system may identify an anterior corneal surface of the eye in the OCT image to determine the z-distance to the eye.

At block 930a, the adjustment system adjusts the optical head to compensate for the xy-offset. For example, the optical head may be adjusted such that the eye center is aligned with the central xy-point of the laser system. The adjustment system may compensate for the xy-offset of the eye according to, e.g., a z-distance to the eye, a previous movement of the eye, and/or a predicted docking region.

At block 932a, the adjustment system moves the optical head towards the eye according to the z-distance to the eye. The computer system may use the OCT image to monitor the z-distance. In some embodiments, the rate at which the optical head is moved towards the eye may change according to the z-distance. For example, for larger z-distances, the rate of movement in the z-direction may be larger, and as the z-distance decreases (e.g., the optical head moves closer to the eye), the rate of movement may decrease. By decreasing the rate of movement in the z-direction as the patient interface comes closer to the eye, the system may be given sufficient time to correct for xy-offset discrepancies before docking/physical contact between the patient interface and the eye.

At block 934a, the computer system determines when the patient interface is in a proper docking position on the eye. The computer system may use the OCT image to determine when the patient interface is in the proper docking position.

At block 936a, the adjustment system ceases moving the optical head when the patient interface is in the proper docking position. The computer system may engage a suction system to fixedly couple the patient interface to the eye. The coupling process may be, e.g., automated, manual, or semi-automated. In an automated process, the computer system may engage the suction system in response to determining that the patient interface is in the proper docking position. In a manual process, the computer system may engage the suction system in response to a user request. In a semi-automated process, the computer system may request that the user confirms that the coupling process may be initiated and engage the suction system in response to confirmation from the user.

At block 920, if the docking mode is a two-step docking process, the method proceeds to block 922b.

At block 922b, the imaging system generates an eye image set that includes images of the eye. The images may include a microscope image of the eye and/or an OCT image of the eye.

At block 924b, the computer system determines an xy-offset of the eye according to the eye image set. For example, the computer system may identify an eye center (which, in some but not all cases, may be indicated by an eye axis) in the microscope image of the eye. The computer may then determine the xy-offset from a difference between the eye center and the central xy-point of the laser system.

At block 926b, the computer system determines a z-distance to the eye according to the eye image set. For example, the computer system may identify an anterior corneal surface of the eye in the OCT image to determine the z-distance to the eye.

At block 930b, the adjustment system adjusts the optical head to compensate for the xy-offset. For example, the optical head may be adjusted such that the eye center is aligned with the central xy-point of the laser system. The adjustment system may compensate for the xy-offset of the eye according to, e.g., a z-distance to the eye, a previous movement of the eye, and/or a predicted docking region.

At block 932b, the adjustment system moves the optical head towards the eye according to the z-distance to the eye. The computer system may use the OCT image to monitor the z-distance. In some embodiments, the rate at which the optical head is moved towards the eye may change according to the magnitude of the z-distance. For example, for larger z-distances, the rate of movement in the z-direction may be larger, and as the z-distance decreases (e.g., the optical head is closer to the eye), the rate of movement may decrease. By decreasing the rate of movement in the z-direction as the patient interface comes closer to the eye, the system may be given sufficient time to correct for xy-offset discrepancies before docking/physical contact between the patient interface and the eye.

At block 934b, the computer system determines when the optical head is in a proper docking position on the patient interface (which is coupled the eye). The computer system may use the OCT image to determine when the optical head is in the proper docking position.

At block 936b, the adjustment system ceases moving the optical head when the optical head is in the proper docking position.

At block 940, the computer system confirms the proper docking position. The computer system may utilize images and/or sensors to confirm the proper docking position.

At block 942, the computer system initiates suction to secure the patient interface to the eye. The computer system may engage a suction system to fixedly couple the patient interface to the eye. The coupling process may be, e.g., automated, manual, or semi-automated. In an automated process, the computer system may engage the suction system in response to determining that the optical head is in the proper docking position. In a manual process, the computer system may engage the suction system in response to a user request. In a semi-automated process, the computer system may request that the user confirms that the coupling process may be initiated and engage the suction system in response to confirmation from the user.

The present disclosure (including the specification, claims, and drawings) includes example embodiments that are intended to aid the reader in understanding the invention and concepts contributed by the inventor to furthering the art and to enable any person skilled in the art to make or use the disclosed embodiments. Modifications (e.g., changes, substitutions, additions, omissions, and/or other modifications) to the embodiments will be readily apparent to those skilled in the art. Accordingly, modifications may be made to the embodiments without departing from the essence of the present disclosure.

In certain instances, modifications may be made to the systems disclosed herein, as apparent to those skilled in the art. For example, parts of a system may be integrated or separated, or an operation of a system may be performed by more, fewer, or other parts. In certain instances, modifications may be made to the methods disclosed herein, as apparent to those skilled in the art. For example, the methods may include more, fewer, or other operations. As another example, certain operations may be optional, combined into fewer operations, or expanded into additional operations. As yet another example, certain operations may be performed in any suitable order or simultaneously.

Furthermore, those skilled in the art will recognize that the present disclosure is not intended to be limited to the example embodiments and that the language of the disclosure is to be accorded the widest scope consistent with the present disclosure. Terms (which may include one or more words) that describe inclusion are generally intended as “open” terms in that they generally do not imply exclusion. For example, the term “including” may be interpreted as “including, but not limited to” or “including at least”; the term “having” may be interpreted as “having, but not limited to” or “having at least”; and the term “comprising” may be interpreted as “comprising, but not limited to” or “comprising at least”, etc.

Additionally, if a specific number is intended, such intent will be explicitly recited in the claim. In the absence of the explicit recitation of a specific number, no such intent is present. If a specific number is explicitly recited, such recitation should be interpreted to mean at least the recited number. For example, the bare recitation of “two Xs”, without other modifiers, may mean “at least two Xs” or “two or more Xs”. Moreover, the use of an indefinite article (e.g., “a” or “an”) or definite article (e.g., “the”) to introduce a noun phrase should not be construed to limit the noun phrase to one, but may be interpreted as an open term “at least one” or “one or more”. This holds even when the same claim includes an open term (e.g., “one or more” or “at least one”) and an indefinite or definite article (e.g., “a” or “an” or “the”).

Moreover, a selection from a list of items should be understood to contemplate a selection of any suitable individual item or any suitable combination of items. For example, the general construction “at least one of A, B, and C” or “one or more of A, B, and C” may include A alone; B alone; C alone; A and B together; A and C together; B and C together; and A, B, and C together. Moreover, any disjunctive term presenting two or more alternative items may be understood to contemplate including one of the items, either of the items, or both items. For example, the general construction “A or B” or “A and/or B” may include A alone, B alone, and A and B together. Additionally, the use of the terms “first,” “second,” “third,” etc. are not necessarily used herein to connote a specific order. For example, the terms “first,” “second,” “third,” etc., may be used to distinguish between different elements.

Additionally, relative terms may be used and understood as typically used in the relevant art. By way of example, relative terms such as “approximately,” “substantially,” “about,” “roughly,” or other similar relative terms mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When such relative terms are used to refer to numbers or values that are part of a range, the term can be used to modify both the minimum and maximum numbers or values.

To aid the Patent Office and readers in interpreting the claims, Applicants note that they do not intend any of the claims or claim elements to invoke 35 U.S.C. §112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the Applicants to refer to structures known to those skilled in the art and is not intended to invoke 35 U.S.C. §112(f).

What is claimed:

1. An ophthalmic system comprising:
a laser system configured to provide a laser beam, the laser system comprising an optical head having an optical axis, the laser system associated with a z-axis and an xy-plane, the z-axis defined relative to the optical axis, the xy-plane defined relative to the z-axis, the xy-plane having a central xy-point;
an adjustment system configured to facilitate a movement of the optical head:
an imaging system configured to generate an eye image set comprising one or more images of an eye; and
a computer system configured to:
determine an xy-offset of the eye according to the eye image set, the xy-offset of the eye relative to the central xy-point;
determine a z-distance to the eye according to the eye image set, the z-distance to the eye corresponding to the z-axis;
instruct the adjustment system to compensate for the xy-offset of the eye; and
instruct the adjustment system to facilitate the movement of the optical head towards the eye according to the z-distance to the eye.

2. The ophthalmic system of claim 1, the computer system configured to determine the xy-offset of the eye according to the eye image set by:
identifying an eye center according to the eye image set; and
determining a difference between the eye center and the central xy-point.

3. The ophthalmic system of claim 2, the computer system configured to identify the eye center according to the eye image set by:
identifying a pupil according to the eye image set; and
identifying a pupil center of the pupil as the eye center.

4. The ophthalmic system of claim 2, the computer system configured to identify the eye center according to the eye image set by:
identifying a lens capsule according to the eye image set; and
identifying a lens capsule center of the lens capsule as the eye center.

5. The ophthalmic system of claim 2, the computer system configured to identify the eye center according to the eye image set by:
identifying a plurality of corneal angles according to the eye image set, a corneal angle of the plurality of corneal angles defined relative to a posterior surface of a cornea of the eye and an anterior surface of an iris of the eye; and
identifying an angle center of the plurality of corneal angles as the eye center.

6. The ophthalmic system of claim 2, the computer system configured to identify the eye center according to the eye image set by:
determining an eye axis according to the eye image set; and
determining the eye center using the eye axis.

7. The ophthalmic system of claim 2, the computer system configured to identify the eye center according to the eye image set by:
receiving a selection of the eye center; and
identifying the selection as the eye center.

8. The ophthalmic system of claim 1, the computer system configured to operate in a docking mode selected from a one-step docking process and a two-step docking process, the one-step docking process starting with a first patient interface coupled to the optical head, the two-step docking process starting with a second patient interface coupled to the eye.

9. The ophthalmic system of claim 8, the computer system configured to:
receive a selection of the docking mode comprising the one-step docking process or the two-step docking process; and
operate according to the selection of the docking mode.

10. The ophthalmic system of claim 8, the computer system configured to:
detect that a patient interface is coupled to the optical head; and
operate according to the one-step docking process.

11. The ophthalmic system of claim 8, the computer system configured to:
detect that no patient interface is coupled to the optical head; and
operate according to the two-step docking process.

12. The ophthalmic system of claim 1, the computer system configured to operate in a docking mode comprising a one-step docking process, the one-step docking process starting with a patient interface coupled to the optical head.

13. The ophthalmic system of claim 12, the computer system configured to:
determine a contact of the patient interface with the eye according to the eye image set; and
instruct the adjustment system to at least reduce the movement of the optical head according to the contact of the patient interface with the eye.

14. The ophthalmic system of claim 1, the computer system configured to operate in a docking mode comprising a two-step docking process, the two-step docking process starting with a patient interface coupled to the eye.

15. The ophthalmic system of claim 14, the computer system configured to:
determine a contact of the optical head with the patient interface according to the eye image set; and
instruct the adjustment system to at least reduce the movement of the optical head according to the contact of the optical head with the patient interface.

16. The ophthalmic system of claim 1, comprising a sensor configured to detect when the optical head is docked to the eye.

17. The ophthalmic system of claim 1, comprising a sensor configured to detect a proximity of the patient interface to the eye.

18. The ophthalmic system of claim 1, comprising a sensor configured to detect a proximity of the optical head to the patient interface.

19. The ophthalmic system of claim 1, comprising a display device configured to display a graphical user interface.

20. The ophthalmic system of claim 19, the graphical user interface configured to:
display a plurality of docking mode options;
receive a selection of a docking mode selected from the plurality of docking mode options; and
notify the computer system of the selection of the docking mode.

21. The ophthalmic system of claim 19, the graphical user interface configured to:
display an automated docking selector;
receive a selection of the automated docking selector; and
notify the computer system of the selection of the automated docking selector.

22. The ophthalmic system of claim 1, comprising a manual actuator configured to:
receive a selection of an automated docking option; and
notify the computer system of the selection of the automated docking option.

23. The ophthalmic system of claim 1, the computer system configured to instruct the adjustment system to compensate for the xy-offset of the eye by:
instructing the adjustment system to compensate for the xy-offset of the eye according to a z-distance to the eye.

24. The ophthalmic system of claim 1, the computer system configured to instruct the adjustment system to compensate for the xy-offset of the eye by:
instructing the adjustment system to compensate for the xy-offset of the eye according to a predicted docking region.

25. The ophthalmic system of claim 1, the computer system configured to instruct the adjustment system to compensate for the xy-offset of the eye by:
instructing the adjustment system to compensate for the xy-offset of the eye according to a previous movement of the eye.
